(19) United States
(12) Patent Application Publication
(10) Pub. No.: US 2024/0307129 A1
(43) Pub. Date: Sep. 19, 2024

(54) NEUROSURGICAL NAVIGATION SYSTEM REFERENCE ARRAY APPARATUS

(71) Applicant: UNIVERSITY OF FLORIDA RESEARCH FOUNDATION, INCORPORATED, Gainesville, FL (US)

(72) Inventors: Travis Eugene MEYER, Gainesville, FL (US); Grzegorz BRZEZICKI, Gainesville, FL (US)

(21) Appl. No.: 18/681,212

(22) PCT Filed: Aug. 4, 2022
(86) PCT No.: PCT/US2022/074522

§ 371 Date: Feb. 5, 2024

Related U.S. Application Data
(60) Provisional application No. 63/255,173, filed on Oct. 13, 2021.
(60) Provisional application No. 63/229,136, filed on Aug. 4, 2021.

(51) Int. Cl. A61B 34/20; A61B 17/00; A61B 17/17; A61B 17/56; A61B 90/00

(52) U.S. Cl. CPC A61B 34/20; A61B 17/1703; A61B 17/1757; A61B 2017/00526; A61B 2017/564; A61B 2034/2072; A61B 2090/3916

(57) ABSTRACT

An apparatus (100), including: a stabilizing bridge (146) including: a C1 attachment (150) configured to be directly attached in a form-fit with a C1 vertebra (124); and a C2 attachment (152) configured to be directly attached in a form-fit with a C2 vertebra (126). The C1 attachment and the C2 attachment are fixed in position relative to each other.

### FIELD OF THE INVENTION

The invention relates to an apparatus that directly connects a navigational reference array to one or more vertebrae. In particular, the apparatus directly connects to two vertebrae and holds the connected vertebrae in fixed positions relative to each other.

### BACKGROUND OF THE INVENTION

First and second cervical vertebral body (C1 and C2, respectively) posterior instrumented fusion is usually reserved for highly unstable fractures or advanced degenerative changes which could lead to spinal cord compression and injury related to instability (excessive motion) between C1 and C2. Given the close proximity to vital structures, accuracy of screw placement is of paramount importance. The current method of C1 and C2 screw placement involves attaching a neurosurgical navigation reference arc of a neurosurgical navigation system to a cranial pin head holder. The reference array and the patient are then scanned so an initial position of the patient's head and other parts of the patient (including the C1 and C2 vertebrae) relative to the reference array are known in a 3D space of the neurosurgical navigation system.

This setup initially provides the position of the C1 and C2 vertebrae with accuracy suitable for C1 and C2 instrumentation. However, the accuracy can deteriorate during the process of screw placement because the process (e.g., drilling, tapping, inserting screws) may move the C1 and/or the C2 vertebrae from their initial positions. This movement is not reflected in the 3D space because the reference array is secured to the patient's head but the C1 and C2 vertebrae are free to move relative to the patient's head. If this relative movement happens, the neurosurgical navigation system will assume the C1 and C2 vertebrae are still in their initial positions when they are not because they have shifted. This can lead to inaccurate instrumentation on the C1 and C2 vertebrae. Hence, there is room in the art for improvement.

### BRIEF DESCRIPTION OF THE DRAWINGS

The invention is explained in the following description in view of the drawings that show:

FIG. 1 is a schematic representation of an example embodiment of a reference array apparatus as used in a neurosurgical navigation system.

FIG. 2 shows a perspective view of an example embodiment of a stabilizing bridge and an example embodiment of a reference arc of the reference array apparatus disclosed herein.

FIG. 3 shows a side perspective view of the embodiment shown in FIG. 2.

FIG. 4 shows an example of the embodiment shown in FIG. 3 with C1 and C2 attachments.

FIG. 5 shows an elongated view of a stabilizing bridge and reference arc secured to C1 vertebra and C2 vertebrae.

FIG. 6 shows a portional view the stabilizing bridge and the reference arc secured to C1 vertebra and the C2 vertebra.

FIG. 7 shows an example embodiment of the reference array apparatus.

### DETAILED DESCRIPTION OF THE INVENTION

Therefore, inventors have devised a unique and innovative custom-made, patient specific, reference array device which attaches to two vertebrae. In an example embodiment, the reference array device attaches to the C1-C2 vertebrae complex, provides temporary C1-C2 fixation, and improves accuracy of intraoperative screw placement. Given a lack of reliable anatomical attachment points and significant anatomical variations in this region of spine, generic fixation devices (spinous process clamps, pins etc.) cannot be reliably used and they do not provide temporary C1-C2 fixation. Hence, a patient-specific attachment is disclosed.

The posterior elements of C1 and C2 are segmented using preoperative scan data (e.g., computed tomography (CT) scan data) to construct a 3D representation of the patient's anatomy. A 3D representation may be a digital model used by a processor and/or may be represented as a 3D image that can be displayed on a monitor or the like. The exact dimensions and shape of the device are then determined based on the 3D representation of the patient's anatomy. The device is designed to be rigid to provide fixed relative reference array spatial relationships but also not to obstruct trajectories needed to place instrumentation. The device attaches to C1 posterior arch and C2 spinous process using standard reconstruction microscrews through planned screw holes in the device. The length and the position of the fixating screws is predefined using the 3D representation of the patient's anatomy. Placement of fixating microscrews does not affect the stability of the spine and moreover this exact attachment area may be decorticated at the end of surgery to promote fusion or removed if C1-2 laminectomy is performed for spinal cord decompression.

Once attached to C1 and C2, and the reference arc is affixed, the surgery progresses as per the current standard of care. The product and any anchoring screws are removed at the end of the screw pilot hole drilling phase and the screws and rods are then attached to the spine as usual. Since the area of the spine where the device is affixed is routinely decorticated or removed towards the end of the procedure, there is no harm or downside to using the device as the anatomy affected by screwing the device to the vertebra is removed as routine standard of care. This device stands to improve operative efficiency and decrease complications by decreasing operative time and increasing accuracy of placement of the screws during C1-C2 fixation.

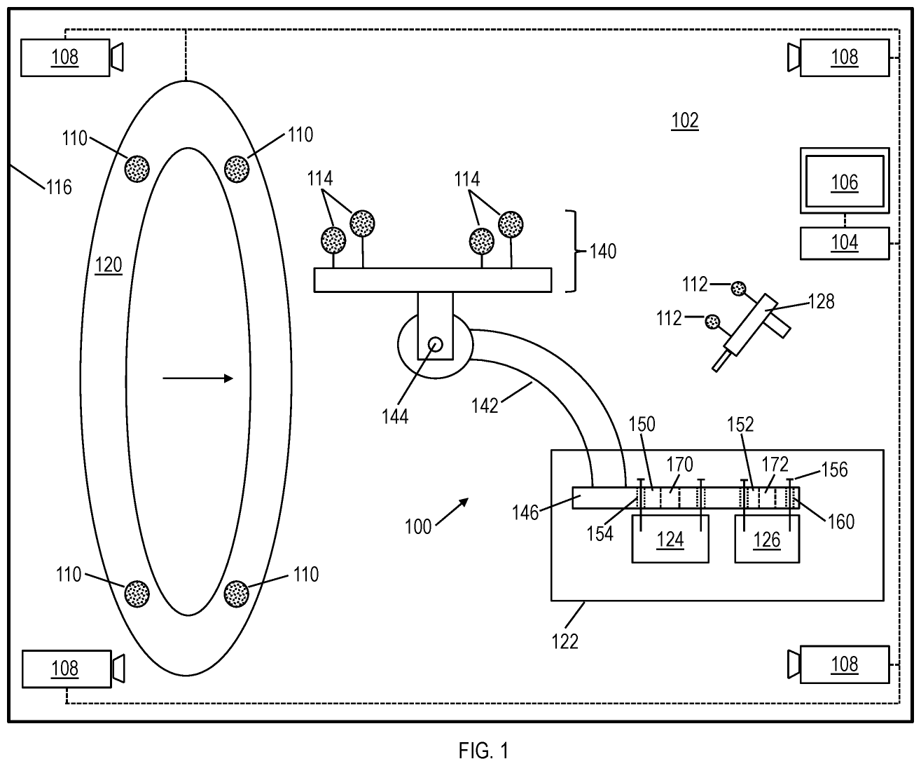

FIG. 1 is a schematic representation of an example embodiment of a reference array apparatus 100 used in a neurosurgical navigation system 102 (e.g., a stereotactic navigation system). The neurosurgical navigation system 102 includes any combination of a controller/processor 104, a monitor 106, cameras 108, scanner reflectors 110, instrumentation reflectors 112, and reference array reflectors 114. The scanner reflectors 110 are disposed on a scanner 120 (e.g., a computed tomography—CT scanner) used to scan a patient 122 having a C1 vertebra 124 and a C2 vertebra 126. The instrumentation reflectors 112 are disposed on one or more instruments 128 (e.g., a drill) used during the procedure. The reference array reflectors 114 are disposed on and also considered part of the reference array apparatus 100 and are used to indicate a position (location and orientation) of the C1 vertebra 124 and the C2 vertebra 126. Any combination of the monitor 106, the cameras 108, and the scanner 120 are in signal communication with the controller 104. The neurosurgical navigation system 102 is set up to operate in an operational space 116 (e.g., a surgical room).

The reference array apparatus 100 includes a reference array 140 secured to a reference arc 142 via a connection 144. The reference arc 142 is, in turn, secured to a stabilizing bridge 146. The stabilizing bridge 146 is secured to and holds the C1 vertebra 124 and the C2 vertebra 126 in fixed positions relative to each other. The stabilizing bridge 146 may include a C1 attachment 150 configured to be directly attached in a form-fit with a C1 vertebra 124, and a C2 attachment 152 configured to be directly attached in a form-fit with a C2 vertebra 126. The C1 attachment 150 and the C2 attachment 150 are configured to be held in a fixed in position relative to each other.

The C1 attachment 150 may include predrilled C1 fixing holes 154 configured to guide respective fixating microscrews 156 (e.g., standard maxillofacial reconstruction microscrews) used to secure the C1 attachment 150, and hence the stabilizing bridge 146, to the C1 vertebra 124. In an example embodiment, the C1 fixing holes 154 may be configured to guide respective fixating microscrews 156 in the posterior arch of the C1 vertebra 124.

The stabilizing bridge 146 may include predrilled C2 fixing holes 160 configured to guide respective fixating microscrews 152 used to secure the C2 attachment 152, and hence the stabilizing bridge 146, to the C2 vertebra 124. In an example embodiment, the C2 fixing holes 160 may be configured to guide respective fixating microscrews 156 in the spinous process of the C2 vertebra 126.

The stabilizing bridge 146 may further include optional C1 instrumentation holes 170 configured to guide the instrumentation 128 relative to the C1 vertebra 124, and optional C2 instrumentation holes 172 configured to guide the instrumentation 128 relative to the C2 vertebra 126. In an example embodiment, the C1 instrumentation holes 170 and the C2 instrumentation holes 172 are designed to guide a drill that drills a pilot hole in the respective vertebra. In example embodiments without C1 instrumentation holes 170 and/or C2 instrumentation holes 172, the shape of the stabilizing bridge 146 is selected to ensure it remains out of the way of the instrumentation during the procedure.

Prior to the procedure, an initial 3D representation of at least a posterior of the C1 vertebra 124 and a posterior of the C2 vertebra 126 of the patient 122 is constructed. The initial 3D representation may be a digital model used by a processor and/or may be represented as a 3D image that can be displayed on a monitor or the like. In an example embodiment, the initial 3D representation is constructed based on one or more computed tomography (CT) scans, though other types of scans may be used and/or the initial 3D representation may be generated via another process. The initial scan reveals an actual shape of a posterior of the C1 vertebra 124 and an actual shape of the posterior of the C2 vertebra 126. In an example embodiment, the initial scan reveals an actual shape of a posterior arch of the C1 vertebra 124 and an actual shape of a spinous process of the C2 vertebra 126. The initial scan may also reveal an initial positional relationship between the C1 vertebra 124 and the C2 vertebra 126.

The C1 attachment 150 is generated as part of the stabilizing bridge 146 and to have a size and shape that form fits with the C1 vertebra 124 based at least on the initial 3D representation of the patient's anatomy. The physician may also base this and any stabilizing bridge 146 decisions on anatomy that is visible to the naked eye. In an example embodiment, the size and shape are a reverse of the actual size and shape of the posterior of the C1 vertebra 126. In an example embodiment, the size and shape are a reverse of the actual size and shape of the posterior arch of the C1 vertebra 124. One or more C1 fixing holes 154 may also be formed in the C1 attachment 150. One or more C1 instrumentation holes 170 may also be formed in the C1 attachment 150. Locations of the C1 fixing holes 154 and the C1 instrumentation holes 170 are selected to avoid anatomical structures such as vertebral arteries, nerve roots, and spinal cord etc. based at least the initial 3D representation, which indicates the positions of these anatomical structures. In an example embodiment, the fixing holes are formed at angles relative to each other to increase the structural integrity of the connection with the vertebra.

The C2 attachment 152 is also generated as part of the stabilizing bridge 146 and to have a size and shape that form fits with the C2 vertebra 126 based at least the initial 3D representation. In an example embodiment, the size and shape are a reverse of the actual size and shape of the posterior of the C2 vertebra 126. In an example embodiment, the size and shape are a reverse of the actual size and shape of the spinous process of the C2 vertebra 126. One or more C2 fixing holes 160 may also be formed in the C2 attachment 152. One or more C2 instrumentation holes 172 may also be formed in the C2 attachment 152. Locations of the C2 fixing holes 156 and the C2 instrumentation holes 172 are likewise selected to avoid the anatomical structures such as vertebral arteries, nerve roots, and spinal cord etc.

The stabilizing bridge 146, its C1 attachment 150, its C2 attachment 152, and any other features may be formed in any suitable manner, including additive and subtractive manufacturing. In an example embodiment, the stabilizing bridge 146 is formed via a 3D printing process. When made as part of a 3D printing process, a 3D model of the stabilizing bridge 146 may be generated first. The 3D model can include any or all of the features of the stabilizing bridge 146 disclosed herein. Further, a 3D model may first be generated as necessary for any other manufacturing process that can be based thereon.

While the size and shape of the C1 attachment 150 and the size and shape of the C2 attachment 152 are dictated by the size and shape of the C1 vertebra 124 and the C2 vertebra 126 respectively, the relative positions (location and orientation) of the C1 attachment 150 and the C2 attachment 152 may or may not be the same as/dictated by the initial positional relationship between the C1 vertebra 124 and the C2 vertebra 126. In an example embodiment, the stabilizing bridge 146 can be formed so that the C1 attachment 150 and the C2 attachment 152 hold the C1 vertebra 124 and the C2 vertebra 126 in an uncorrected stabilized positional relationship that is the same as the initial positional relationship. However, in an alternate example embodiment, the stabilizing bridge 146 can be formed so that the C1 attachment 150 and the C2 attachment 152 hold the C1 vertebra 124 and the C2 vertebra 126 in a corrected stabilized positional relationship that is different than the initial positional relationship. The corrected stabilized positional relationship can be determined by, for example, a physician, and can represent a degree of reduction/correction in the positional relationship between the C1 vertebra 124 and the C2 vertebra. It is also possible to create multiple stabilizing bridges 146 in preparation for the procedure, where each stabilizing bridge 146 represents a unique stabilized positional relationship. For example, preparation may include forming any combination of a stabilizing bridge 146 that is configured to hold the C1 vertebra 124 and the C2 vertebra 126 in the uncorrected stabilized positional relationship, and one or more stabilizing bridges 146, where each stabilizing bridge 146 is configured to position the C1 vertebra and the C2 vertebra relative to each other in a unique corrected stabilized positional relationship. Providing multiple stabilizing bridges 146, where each stabilizing bridge 146 represents a unique positional relationship, provides flexibility for the physician in making decisions during the procedure.

In addition, the stabilizing bridge 146, and hence the C1 connection 150 and the C2 connection 152, can be made from a material that can be further formed during the procedure. For example, the stabilizing bridge 146 can be made of a plastic material that the physician can grind during the procedure as the physician sees fit using instrumentation that is available during the procedure to create a desired fit with either or both the C1 vertebra 124 and the C2 vertebra 126. Similarly, such a workable/machinable material enables the physician to create fixing holes in different locations than originally planned if the physician deems it necessary.

The reference arc 142 may be formed as one piece with the stabilizing bridge 146 to create a fixed positional relationship therebetween (e.g., via a manufacturing process such as 3D printing). Alternately, the reference arc 142 may be formed separately and secured to the stabilizing bridge 146 via any suitable means, such as mechanically via e.g., fasteners.

The reference arc 142 provides a link between the stabilizing bridge 146 and the connection 144 with the reference array 140. In addition, the reference arc 142 positions the reference array 140 remote from the surgery site so that the reference array 140 does not interfere with the procedure. In an example embodiment, the reference arc 142 positions the reference array 140 in a location superior to the C1 vertebra 124. An example location is proximate the posterior side of the patient's head. The connection 144 can be any type suitable for use with any reference array 140 and in an example embodiment is a pivoting connection suitable to pivot the reference array 140 about the connection 144.

The stabilizing bridge 146, the reference arc 142, and the reference array 140 are secured together (at any time), the C1 vertebra 124 and the c2 vertebra 126 are surgically exposed during the procedure, the C1 connection 150 is secured to the C1 vertebra 124 via fixating microscrews 156, and the C2 connection is secured to the C2 vertebra via fixating microscrews 156. Once the reference array apparatus 100 is so assembled and secured to the patient 122, and once the patient 122, the scanner 120, and neurosurgical navigation system 102 are co-located in the operational space 116, the arrangement shown in FIG. 1 is reached.

A combined 3D representation of the patient 122 and the reference array apparatus 100 is generated using the scanner 120 and the processor 104 (or other processor) and is suitable for use by the neurosurgical navigation system 102. The combined 3D representation may be a digital model used by a processor and/or may be represented as a 3D image that can be displayed on a monitor or the like. In an example embodiment, the scanner is a computed tomography (CT) scanner, though other types of scanners are possible. To do this, the cameras 108 continually/repeatedly identify the scanner reflectors 110 and the reference array reflectors 114 and register their positions in a 3D space. The scanner 120 scans the patient 122 and the reference array apparatus 100. By virtue of knowing the positions of the scanner reflectors 110 and the reference array reflectors 114 in the 3D space during the scan, the position of the patient 122 and the patient's anatomical structures, including the C1 vertebra 124, the C2 vertebra 126, and the vertebral arteries, nerve roots, and spinal cord etc. are likewise known in the 3D space.

The neurosurgical navigation system 102 continues to register the various reflectors to ensure the 3D space accurately represents the actual locations of the patient 122 and the scanner 120. Any instrumentation 128 present in the operational space 116 having the instrumentation reflectors 112 will similarly be registered in the 3D space. Importantly, included in the information regarding the instrumentation 128 is information regarding the position of items like a tip of a drill bit that enters the patient 122 as part of the procedure.

The 3D space can be displayed as a 3D representation of the 3D space on the monitor 106 or the like. As such, the 3D representation that is visible to the physician includes the positions of the internal anatomy of the patient that are not visible to the naked eye (from the combined 3D representation) as well as the positions (location and orientation) of the instrumentation 128. The physician can use this information to ensure the instrumentation 128 does not reach anatomical structures that can be damaged and ensure proper fusion is achieved. For example, the physician can watch the 3D representation of the drill bit as it enters a vertebra on the monitor and thereby guide the drill bit to avoid the internal anatomical structures that are also displayed on the monitor 106. Accuracy of the registration of the patient 122 and the patient's anatomical structures can be verified by touching known anatomical structures with a navigational probe recognized by the neurosurgical navigation system 102 and ensuring that the location of the actual touch is accurately reflected in the 3D representation.

It is important to note that continued registration process is different from generating the combined 3D representation of the patient 122 and the reference array apparatus 100. During the continued registrations, which happens between construction of combined 3D representations (if more than one combined 3D representation is constructed), the cameras can only register and update the positions of the reflectors and associated items such as the reference array 140 and instrumentation 128. During the construction of the combined 3D representation of the reference array 140 and the patient 122, one or more spatial relationships are established between the reference array 140 and the patient 122 and associated anatomy. The 3D space assumes these spatial relationships remain unchanged during the continued registrations and will display the location of the patient 122 and associated anatomy relative to the reference array 140 based at least these initially established spatial relationships.

Because the reference array apparatus 100 is secured directly to the C1 vertebra 124 and the C2 vertebra 126, the actual spatial relationship between the reference array 140 and the C1 vertebra 124 and the C2 vertebra 126 will not change. Because the neurosurgical navigation system 102 is constantly registering the location of the reference array 140, the neurosurgical navigation system 102 will know whenever the reference array 140 has moved and it will reflect this movement in the 3D representation. Since the neurosurgical navigation system 102 assumes the spatial relationship between the reference array 140 and the C1 vertebra 124 and the C2 vertebra 126 does not change, the location of the C1 vertebra 124 and the C2 vertebra 126 will be updated on the 3D representation to reflect the initially established spatial relationship. Since the reference array 140 is connected to the C1 vertebra 124 and the C2 vertebra 126 in a fixed positional relationship, the actual spatial relationship between the reference array 140 and the C1 vertebra 124 and the C2 vertebra 126 likewise did not change. Hence, the represented position of the C1 vertebra 124 and the C2 vertebra 126 shown on the 3D representation will match the actual position of the C1 vertebra 124 and the C2 vertebra 126. In short, the reference array apparatus 100 ensures the actual positional relationship between the reference array 140 and the C1 vertebra 124 and the C2 vertebra 126 never changes. This way, the neurosurgical navigation system 102 is made right when it assumes the positional relationship between the reference array 140 and the C1 vertebra 124 and the C2 vertebra 126 never changes. As such, the physician will always be relying on/viewing accurate positional information.

In contrast, in the prior art, the reference array would be secured to the patient's head. Here again, the initial positions of the C1 vertebra 124 and C2 vertebra 126 were known from the combined 3D representation of the patient and reference array (attached to the patient's head). However, the neurosurgical navigation system 102 only continually registers the position of the reference array, not the position of the patient and associated anatomy. Since the reference array is secured to the patient's head, and because the C1 vertebra 124 and C2 vertebra 126 can move relative to each other and relative to the patient's head, the neurosurgical navigation system 102 would not know whether either or both the C1 vertebra 124 and C2 vertebra 126 have moved. This is because the neurosurgical navigation system 102 would assume the initially established positional relationship between the reference array 140 and the C1 vertebra 124 and C2 vertebra 126 always remains the same. After movement of the C1 vertebra 124 and/or C2 vertebra 126, a new positional relationship would be established between the reference array 140 and the C1 vertebra 124 and C2 vertebra 126. However, the neurosurgical navigation system 102 would not know this. Hence, the 3D representation would inaccurately reflect the position of the C1 vertebra 124 and C2 vertebra 126. Since accurate position information is used to avoid anatomical damage as well as improve accuracy, this scenario is problematic.

Once the neurosurgical navigation system 102 displays the 3D representation, the physician can proceed to use the instrumentation 128. In an example embodiment in which the stabilizing bridge 146 does not include C1 instrumentation holes 170 and the C2 instrumentation holes 172, the surgeon will establish the entry points and desired trajectories for the screws based on navigation according to current standards of care. The intended trajectories can be saved in the navigation system and then compared to actual screw placement observed on confirmatory scan for quality assurance.

In an alternate example embodiment, the instrumentation 128 includes a drill and the C1 instrumentation holes 170 and the C2 instrumentation holes 172 act as guides having respective trajectories that matches preplanned trajectories. Positioning the drill bit in the C1 instrumentation holes 170 and the C2 instrumentation holes 172 would thereby align the drill bit with the preplanned trajectories and aid the physician in drilling the pilot holes. Here again, at any time such as after each screw/instrument is placed, or any other time at the discretion of the physician, the accuracy of the 3D representation can be verified by touching the known anatomical structures with a navigational probe and ensuring that the location of the actual touch is accurately reflected in the 3D representation. Similarly, additional combined 3D representations can be generated at any time to ensure the accuracy of placement of the screws etc. For example, the combined 3D representations can compare the actual location of the instrumentation to the planned preplanned trajectories. In this alternate example embodiment, if multiple stabilizing bridges 146 are prepared, and if the preplanned trajectories vary depending on which stabilizing bridge 146 is used, then the physician would need to advise the neurosurgical navigation system 102 which stabilizing bridge 146 has been selected so that the associated preplanned trajectories can be displayed.

Returning to the example embodiment in which the stabilizing bridge 146 does not include C1 instrumentation holes 170 and the C2 instrumentation holes 172, upon completion of drilling the pilot holes and placement of the C1 vertebra 124 and C2 vertebral 126 screws, the fixating microscrews 156 and the stabilizing bridge 146 are removed. Rods are attached to the screw heads and secured with set screws per specific manufacturer's instructions. Since the anatomy of the C1 vertebra 124 and the C2 vertebra 126 where the fixating microscrews 156 enter is routinely decorticated or removed towards the end of the procedure as routine standard of care, there is no harm or downside to using the fixating microscrews 156 to attach the stabilizing bridge 146.

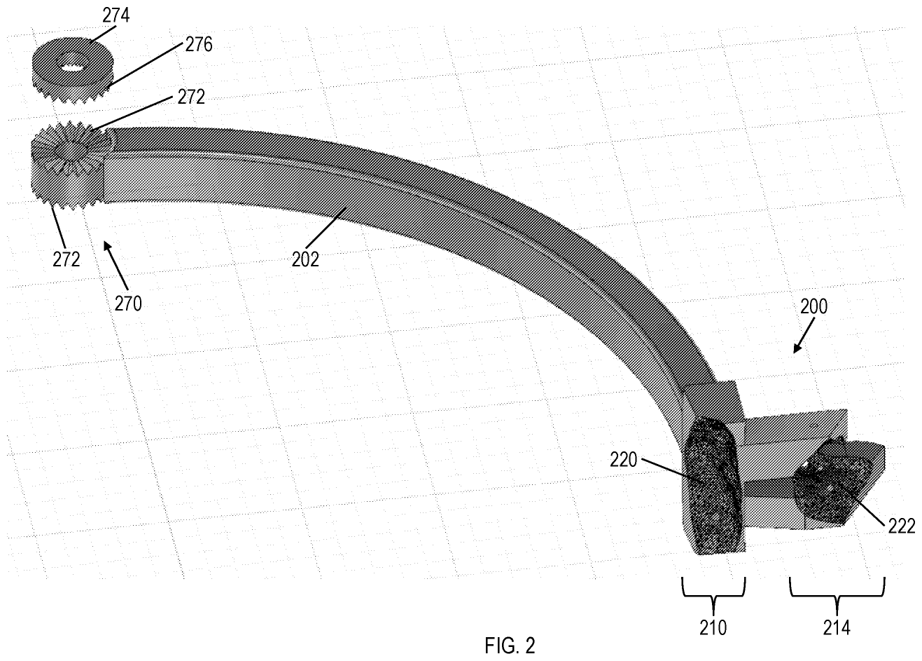

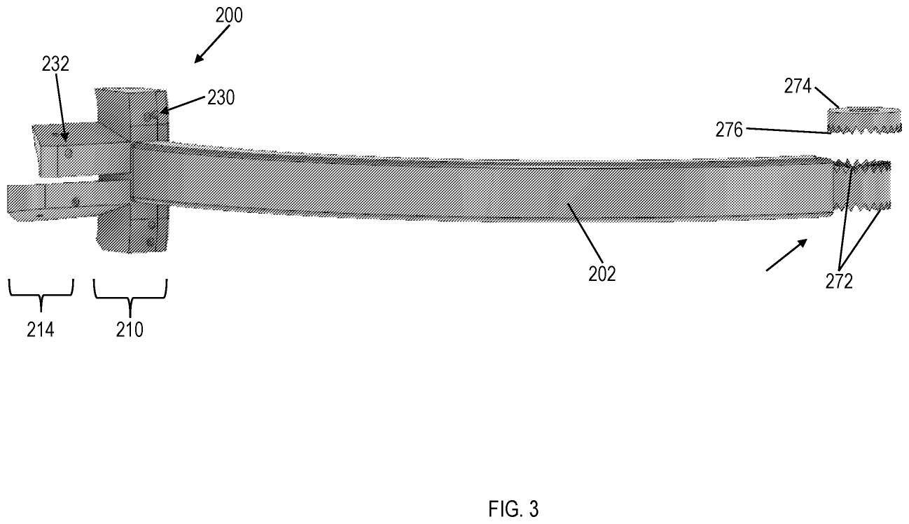

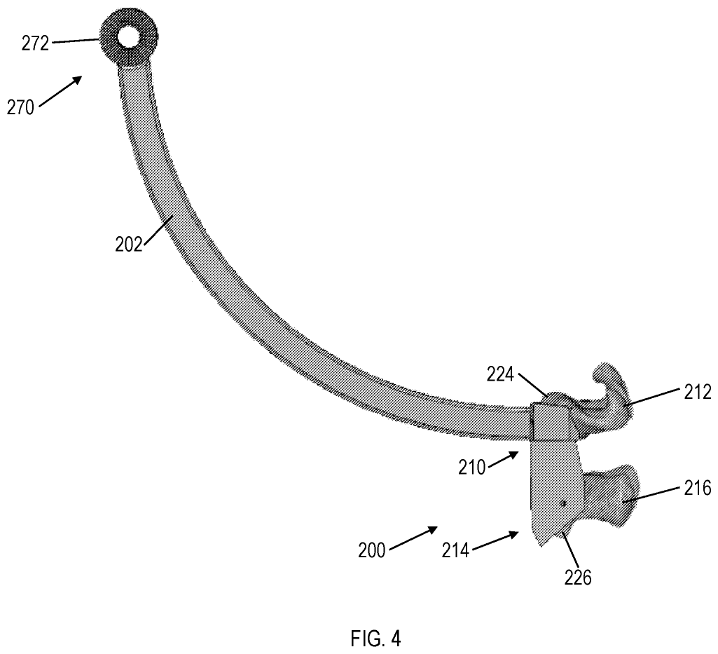

FIG. 2 to FIG. 6 show an example embodiment of the stabilizing bridge 200, the reference arc 202, and the reference array 204 of the reference array apparatus 206. The stabilizing bridge 200 includes the first attachment 210 configured to be directly attached in a form-fit with the first vertebra 212; and the second attachment 214 configured to be directly attached in a form-fit with the second vertebra 216. The first attachment 210 and the second attachment 214 are fixed in position relative to each other. In an example embodiment, the first attachment 210 is a C1 attachment 210, the second attachment 214 is a C2 attachment 216, the first vertebra 212 is a C1 vertebra 212, and the second vertebra 216 is the C2 vertebra 216.

In an example embodiment, the C1 attachment 210 includes a C1 attachment size and shape 220 that are a reverse of an actual size and shape of the C1 vertebra 212, and the C2 attachment 214 includes a C2 attachment size and shape 222 that are a reverse of an actual size and shape of the C2 vertebra 216. In an example embodiment, the C1 attachment size and shape are a reverse of an actual size and shape of a posterior arch 224 of the C1 vertebra 212, and the C2 attachment size and shape are a reverse of an actual size and shape of a spinous process 226 of the C2 vertebra 216.

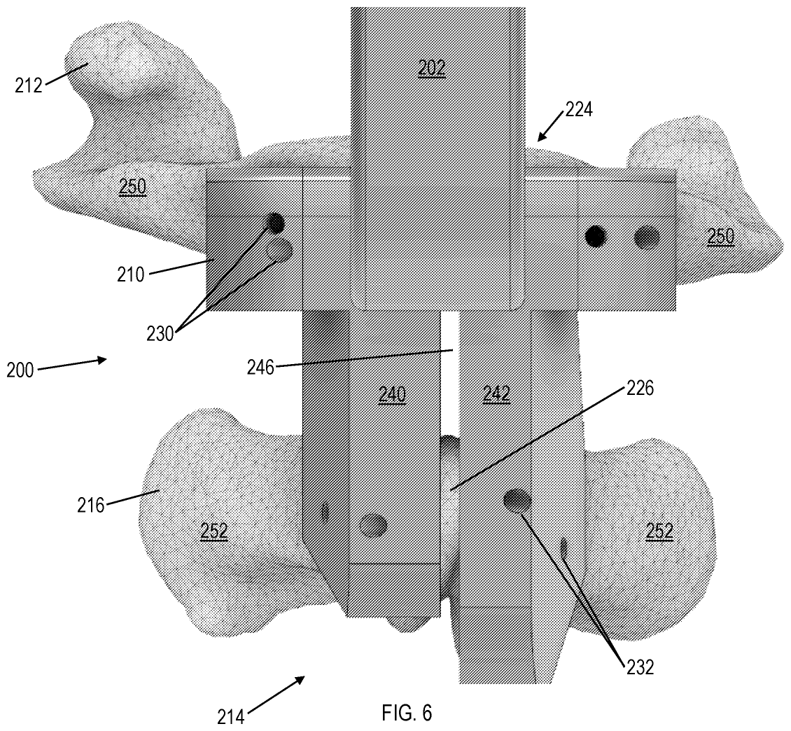

In an example embodiment, the C1 attachment 210 includes one or more C1 fixing holes 230 configured to guide respective fixating microscrews into the C1 vertebra 200, and the C2 attachment 214 includes one or more C2 fixing holes 232 configured to guide respective fixating microscrews into the C2 vertebra 216. In an example embodiment, the C1 fixing holes 230 are configured to guide the respective fixating microscrews into the posterior arch 224 of the C1 vertebra 212, and the C2 fixing holes 232 are configured to guide the respective fixating microscrews into the spinous process 226 of the C2 vertebra 216. As can be seen at least in FIG. 6, in an example embodiment, the C1 fixing holes 230 are configured to guide respective fixating microscrews into the C1 vertebra 212 at different angles relative to each other, and the C2 fixing holes 232 are configured to guide respective fixating microscrews into the C2 vertebra 216 at different angles relative to each other.

In an example embodiment, the stabilizing bridge 200 is composed of a material that can be readily machined using instrumentation available during the procedure. An example material that can be machined, not meant to be limiting, is plastic. A plastic stabilizing bridge 200 can be reshaped via, for example, grinding and/or drilling to achieve a desired outcome. Such adjustment during the procedure is not possible in prior art stabilizing bridges made of materials like steel that cannot be modified using instrumentation available during the procedure. In addition, when the stabilizing bridge 200 is composed of plastic, the stabilizing bridge 200 can be manufactured via additive manufacturing techniques such as via 3D printing processes. This decreases costs and other complexities associated with manufacturing custom stabilizing bridges experienced using prior art techniques.

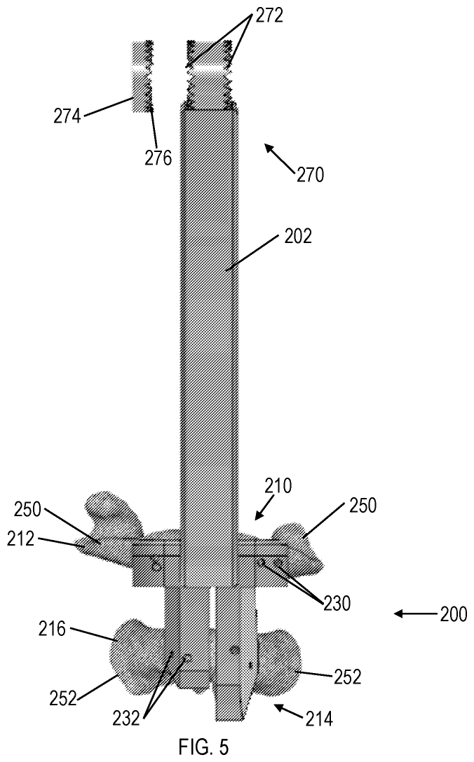

As can be seen in FIG. 5 and FIG. 6, in an example embodiment, the C1 attachment 210 forms a crossbar and the C2 attachment 214 extends transverse to the C1 attachment 210. Hence, the stabilizing bridge 200 can take a shape that resembles a T-shape. In this example embodiment, the C2 attachment 214 includes a first prong 240 and a second prong 242, both extending transverse to C1 attachment 210 and separated from each other by a gap 244. The first prong 240 and the second prong 242 are configured to receive the spinous process 226 of the C2 vertebra 216 therebetween. The lengths of the prongs 240, 242 and the gap 244 between the prongs 240, 242 are dependent on the specific anatomy of the spinous process 226 of the C2 vertebra 216 and a position of the spinous process 226 of the C2 vertebra 216 in relation to the posterior arch 224 of the C1 vertebra 212. In addition, the C2 attachment 214 and prongs 240, 242 thereof are designed in a way to provide tight fit but not to obstruct screw trajectories. The length of the prongs 240, 242 is limited in the way that the prongs 240, 242 do not extend beyond an inferior margin of C2 spinous process.

In the example embodiment shown, the T-shape of the stabilizing bridge 200 does not cover the C1 lateral masses 250 or the C2 lateral masses 252. This provides the physician with the working space required for the instrumentation to drill the pilot holes and place screws.

In an example embodiment, the stabilizing bridge further includes C1 instrumentation holes (not shown) configured to guide an instrumentation drill into the C1 vertebra 212, and C2 instrumentation holes (not shown) configured to guide an instrumentation drill into the C2 vertebra 216.

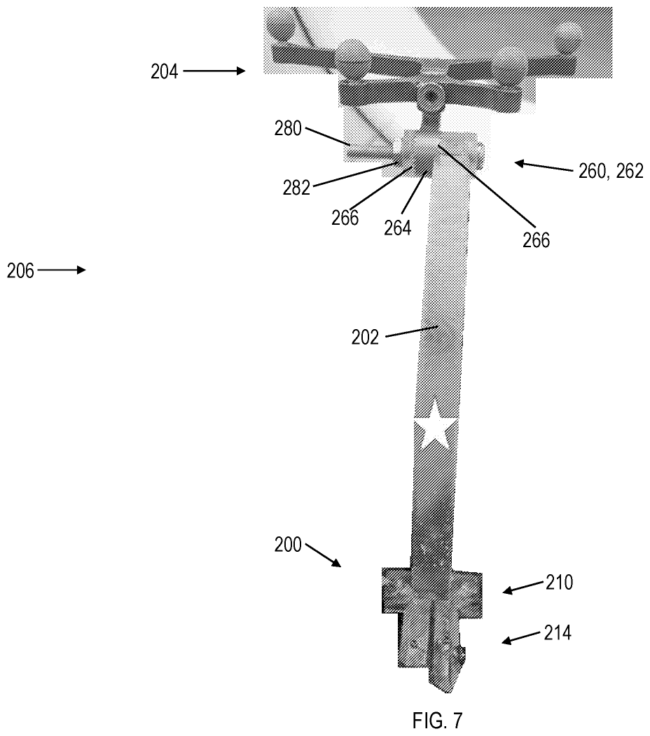

As can be seen in FIG. 7, the reference arc 202 is configured to secure the reference array 204 of the neurosurgical navigation system in a fixed position relative to the stabilizing bridge 200. In an example embodiment, the reference arc 202 is configured to secure the reference array 204 in a position superior to a location of the C1 vertebra 212 and the C2 vertebra 126. This remote placement leaves room for the physician to operate. In an example embodiment, a combined stabilizing bridge and reference arc 3D model includes the 3D model of the stabilizing bridge 200 and a 3D model of the reference arc 202, and the reference arc 202 is generated with the stabilizing bridge 200. In an example embodiment, the stabilizing bridge 200 and the reference arc 202 are 3D printed together as a monolithic body. In an example embodiment, the monolithic body is formed during a 3D printing operation.

Fixing the reference arc 202 in position is achieved by a connection 260. In this example embodiment, the reference array 204 includes a detent arrangement 262. The detent arrangement 262 includes a reference array connector 264 with an annular array of teeth 266 on each side of the reference array connector 264. The reference array connector 264 is sandwiched between an end 270 of the reference arc 202 that includes annular arrays of teeth 272 that intermesh with the annular array of teeth 266 on one side of the reference array connector 264, and a reference arc nut 274 that also includes an annular array of teeth 276 that intermesh with the annular array of teeth 266 on another side of the reference array connector 264. A bolt 280 passes through the detent arrangement 262. Tightening a nut 282 on the bolt 280 tightens the detent arrangement 262, which secures the reference array 204 in any one of a variety of fixed positions relative to the reference arc 202. The reference arc 202 is held in a fixed position relative to the stabilizing bridge 200. As such, the reference array 204 is held in any one of the variety of fixed positions relative to the stabilizing bridge 200.

The reference array apparatus disclosed herein provides a simple, extremely effective, flexible way of fixing the C1 vertebra and the C2 vertebra relative to each other during a surgical procedure. This, in turn, increases accuracy of and flexibility during the surgical procedure. Hence, the reference array apparatus represents an improvement in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The invention claimed is:

1. An apparatus, comprising:
a stabilizing bridge comprising: a first attachment configured to be directly attached in a form-fit with a first vertebra; and a second attachment configured to be directly attached in a form-fit with a second vertebra; wherein the first attachment and the second attachment are fixed in position relative to each other.

2. The apparatus of claim 1, wherein the first attachment comprises a C1 attachment, wherein the second attachment comprises a C2 attachment, wherein the first vertebra comprises a C1 vertebra, and wherein the second vertebra comprises a C2 vertebra.

3. The apparatus of claim 2, wherein the stabilizing bridge further comprises C1 instrumentation holes configured to guide an instrumentation drill into the C1 vertebra.

4. The apparatus of claim 3, wherein the stabilizing bridge further comprises C2 instrumentation holes configured to guide the instrumentation drill into the C2 vertebra.

5. The apparatus of claim 2, wherein the C1 attachment comprises a C1 attachment size and shape that are a reverse of an actual size and shape of the C1 vertebra.

6. The apparatus of claim 5, wherein the C1 attachment size and shape are a reverse of an actual size and shape of a posterior arch of the C1 vertebra.

7. The apparatus of claim 6, further comprising a plurality of C1 fixing holes configured to guide respective fixating microscrews into the posterior arch of the C1 vertebra.

8. The apparatus of claim 7, wherein the plurality of C1 fixing holes configured to guide respective fixating microscrews into the posterior arch at different angles relative to each other.

9. The apparatus of claim 2, wherein the C2 attachment comprises a C2 attachment size and shape that are a reverse of an actual size and shape of the C2 vertebra.

10. The apparatus of claim 9, wherein the C2 attachment size and shape are a reverse of an actual size and shape of a spinous process of the C2 vertebra.

11. The apparatus of claim 10, further comprising a plurality of C2 fixing holes are configured to guide respective fixating microscrews into the spinous process of the C2 vertebra.

12. The apparatus of claim 11, wherein the plurality of C2 fixing holes are configured to guide respective fixating microscrews into the spinous process at different angles relative to each other.

13. The apparatus of claim 2, wherein the C1 attachment forms a crossbar and wherein the C2 attachment extends transverse to the C1 attachment.

14. The apparatus of claim 13, wherein the C1 attachment is configured to be secured to a posterior arch of the C1 vertebra.

15. The apparatus of claim 14, wherein the C2 attachment comprises two prongs extending transverse to the C1 attachment and separated from each other by a gap.

16. The apparatus of claim 15, wherein the two prongs are configured to receive a spinous process of the C2 vertebra therebetween.

17. The apparatus of claim 2, further comprising a reference arc configured to secure a navigation reference array of a neurosurgical navigation system in a fixed position relative to the stabilizing bridge and configured to place the navigation reference array in a position superior to a location of the C1 vertebra and the C2 vertebra.

18. The apparatus of claim 2, wherein the stabilizing bridge comprises a plastic material.

19. The apparatus of claim 17, further comprising the navigation reference array securable by the apparatus in the fixed position relative to the stabilizing bridge and suitable for use with a 3D image surgical guidance system.

20. The apparatus of claim 17, further comprising the neurosurgical navigation system, the neurosurgical navigation system comprising:
an imaging system configured to register the navigation reference array in a 3D space; configured to determine a position of the C1 vertebra and a position of the C2 vertebra in the 3D space; and configured to register a position of a tool relative to the position of the C1 vertebra and the position of C2 vertebra in the 3D space.

21. An apparatus, comprising:
a stabilizing bridge comprising: a C1 attachment configured to be directly attached in a form-fit with a C1 vertebra; and a C2 attachment configured to be directly attached in a form-fit with a C2 vertebra; wherein the C1 attachment and the C2 attachment are fixed in position relative to each other; and
a reference arc configured to be secured in a fixed position relative to the stabilizing bridge, configured to secure a navigation reference array in a fixed position relative to the stabilizing bridge, and configured to place the navigation reference array in a position superior to a location of the C1 vertebra and the C2 vertebra.

22. The apparatus of claim 21, wherein the C1 vertebra comprises a C2 attachment size and shape that are a reverse of an actual size and shape of a posterior arch of the C1 vertebra.

23. The apparatus of claim 22, wherein the C2 attachment size and shape are a reverse of an actual size and shape of a spinous process of the C2 vertebra.

24. The apparatus of claim 23, wherein the C2 attachment size and shape are configured to receive the spinous process of the C2 vertebra therein.

25. The apparatus of claim 23, further comprising a plurality of fixing holes configured to guide respective fixating microscrews into the posterior arch of the C1 vertebra and into the spinous process of the C2 vertebra.

26. The apparatus of claim 21, wherein the stabilizing bridge comprises a plastic material.

27. A method, comprising:
generating an initial 3D representation of at least a posterior of a C1 vertebra and a posterior of a C2 vertebra of a patient; and
generating a stabilizing bridge comprising:
a C1 attachment comprising a size and shape that are a reverse of an actual size and shape of the posterior of the C1 vertebra based at least the initial 3D representation;
a C2 attachment comprising a size and shape that are a reverse of an actual size and shape of the posterior of the C2 vertebra based at least the initial 3D representation;
wherein the C1 attachment and the C2 attachment are in fixed positions relative to each other.

28. The method of claim 27, wherein the stabilizing bridge further comprises C1 instrumentation holes configured to guide an instrumentation drill into the C1 vertebra, wherein locations of the C1 instrumentation holes are selected based at least the initial 3D representation.

29. The method of claim 28, wherein the stabilizing bridge further comprises C2 instrumentation holes configured to guide the instrumentation drill into the C2 vertebra, wherein locations of the C2 instrumentation holes are selected based at least the initial 3D representation.

30. The method of claim 27, wherein the stabilizing bridge is one of a plurality of stabilizing bridges, and wherein each stabilizing bridge of the plurality of stabilizing bridges is configured to position the C1 vertebra and the C2 vertebra relative to each other in a unique positional relationship.

31. The method of claim 30, wherein the initial 3D representation indicates an initial positional relationship of the C1 vertebra relative to the C2 vertebra, and wherein each positional relationship represents a respective level of reduction/correction from the initial positional relationship.

32. The method of claim 27, wherein the actual size and shape of the posterior of the C1 vertebra comprise a size and shape of a posterior arch of the C1 vertebra.

33. The method of claim 32, wherein the stabilizing bridge further comprises C1 fixing holes configured to guide respective fixating microscrews into the posterior arch of the C1 vertebra, wherein locations of the C1 fixing holes are selected based at least the initial 3D representation.

34. The method of claim 33, wherein the C1 fixing holes are disposed at different angles relative to each other.

35. The method of claim 32, wherein the actual size and shape of the posterior of the C2 vertebra comprise a size and shape of a spinous process of the C2 vertebra.

36. The method of claim 35, wherein the stabilizing bridge further comprises C2 fixing holes configured to guide respective fixating microscrews into the spinous process of the C2 vertebra, wherein locations of the C2 fixing holes are selected based at least the initial 3D representation.

37. The method of claim 36, wherein the C2 fixing holes are disposed at different angles relative to each other.

38. The method of claim 35, wherein the C1 attachment forms a crossbar and wherein the C2 attachment extends transverse to the C1 attachment.

39. The method of claim 38, wherein the C2 attachment comprises two prongs extending transverse to the C1 attachment and separated from each other by a gap.

40. The method of claim 39, wherein the two prongs are configured to receive the spinous process of the C2 vertebra therebetween.

41. The method of claim 35, further comprising generating the stabilizing bridge via an additive manufacturing process.

42. The method of claim 41, wherein the additive manufacturing process comprises 3D printing.

43. The method of claim 35, further comprising providing a reference arc configured to secure a navigation reference array of a neurosurgical navigation system in a fixed position relative to the stabilizing bridge.

44. The method of claim 43, further comprising generating the reference arc and the stabilizing bridge as part of a single, monolithic body.

45. The method of claim 43, wherein the reference arc is configured to place the navigation reference array in a position superior to a location of the C1 vertebra and the C2 vertebra.

46. The method of claim 43, further comprising:
assembling the navigation reference array; the reference arc; and the stabilizing bridge together;
securing the C1 attachment to the posterior arch of the C1 vertebra;
securing the C2 attachment to the spinous process of the C2 vertebra; and
generating a combined 3D representation of the patient together with the navigation reference array suitable for use by the neurosurgical navigation system.

47. The method of claim 46, further comprising refining the size or shape of at least one of the C1 attachment and the C2 attachment to create a desired fit.

48. The method of claim 43, further comprising:
forming C1 fixing holes in the stabilizing bridge configured to guide respective fixating microscrews into the C1 vertebra, wherein locations of the C1 fixing holes are selected based at least the initial 3D representation; and
forming C2 fixing holes in the stabilizing bridge configured to guide respective fixating microscrews into the C2 vertebra, wherein locations of the C2 fixing holes are selected based at least the initial 3D representation.

49. The method of claim 48, further comprising:
assembling the navigation reference array; the reference arc; and the stabilizing bridge together;
securing the C1 attachment to the posterior arch of the C1 vertebra via C1 microscrews installed through the C1 fixing holes;
securing the C2 attachment to the spinous process of the C2 vertebra via C2 microscrews through the C2 fixing holes; and
generating a combined 3D representation of the patient together with the navigation reference array suitable for use by the neurosurgical navigation system.

50. The method of claim 49, further comprising:
drilling pilot holes and placing screws in the C1 vertebra; and
drilling pilot holes and placing screws in the C2 vertebra.

51. The method of claim 49, further comprising:
drilling pilot holes and placing screws in the C1 vertebra using a tool guided by the neurosurgical navigation system, wherein the guidance is based at least the combined 3D representation of the patient together with the navigation reference array; and
drilling pilot holes and placing screws in the C2 vertebra using a tool guided by the neurosurgical navigation system, wherein the guidance is based at least the combined 3D representation of the patient together with the navigation reference array.
